(19) United States
(12) Patent Application Publication
(10) Pub. No.: US 2025/0319019 A1
(43) Pub. Date: Oct. 16, 2025

(54) BUCCOMUCOADHESIVE PATCH-BASED DRUG DELIVERY SYSTEM FOR TACROLIMUS

(71) Applicants: Smita PATTANAIK, Chandigarh (IN); Ashish SHARMA, Chandigarh (IN); Vandita KAKKAR, Chandigarh (IN)

(72) Inventors: Smita PATTANAIK, Chandigarh (IN); Ashish SHARMA, Chandigarh (IN); Vandita KAKKAR, Chandigarh (IN)

(21) Appl. No.: 19/110,221

(22) PCT Filed: Sep. 9, 2023
(86) PCT No.: PCT/IB2023/058948

§ 371 Date: Mar. 10, 2025

(30) Foreign Application Priority Data
Sep. 10, 2022 (IN) 202211051783

(51) Int. Cl. A61K 9/00; A61K 31/436; A61K 47/10; A61K 47/38

(52) U.S. Cl. CPC A61K 9/006; A61K 31/436; A61K 47/10; A61K 47/38

(57) ABSTRACT

The present disclosure relates to buccal mucoadhesive patch formulation of tacrolimus loaded in the solid lipid nanoparticles and using the polymeric carrier matrix and pharmaceutically acceptable additives to deliver tacrolimus through the trans buccal route to the systemic circulation. The overall exposure to tacrolimus with the tacrolimus buccal mucoadhesive patch formulation of present invention is considerably higher compared to the conventional oral administration for equivalent dose. It provides an immediate and controlled release of tacrolimus by avoiding the first pass metabolism and providing a steady concentration of tacrolimus in the systemic circulation.

### FIELD OF THE INVENTION

The present disclosure relates to the technical field of pharmaceutical formulation. In particular, the present disclosure relates to a buccomucoadhesive patch formulation of tacrolimus (BAPTac) for transbuccal administration. The tacrolimus buccal mucoadhesive patch formulation of the present invention provides an efficient, immediate as well as controlled release of tacrolimus by avoiding the first-pass metabolism and providing a stable concentration of tacrolimus in the systemic circulation.

### BACKGROUND OF THE INVENTION

Tacrolimus is a calcineurin-inhibitor immunosuppressant drug used mainly for the prophylaxis of organ rejection in patients receiving allogeneic liver, kidney or heart transplants. Tacrolimus is a macrolide immunosuppressant produced by Streptomyces tsukubaensis and inhibits T-lymphocyte activation. Chemically, tacrolimus is designated as [3S[3R*[E(1S*,3S*,4S*)],4S*,5R*,8S*,9E,12R*,14R*,15S*,16R*,18S*,19S*, 26aR*]]5,6,8,11,12,13,14,15,16,17,18,19,24,25,26,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]oxaazacyclotricosine-1,7,20,21(4H,23H)-tetrone, monohydrate.

Tacrolimus is a BCS class-II drug and highly lipophilic compound with a molecular weight of about 804 kD. It has poor and variable bioavailability due to large interindividual variability in intestinal absorption, with an average of 25%. The immediate-release formulations of tacrolimus are the predominant form of the drug and are used by 99% of renal transplant patients. However, there are several clinical scenarios where oral drug delivery is a challenge. Two such scenarios are:
Scenario 1: Patients with altered consciousness on mechanical ventilation or oropharyngeal dysphagia who are on nasogastric (NG) tube for their nutritional requirement.
Scenario 2: Patients with intestinal obstruction or paralytic ileus (post-operative or non-postoperative): NG tubes are placed in these patients for suction and drainage of the gastric secretion and to administer tacrolimus.

In both situations, the tacrolimus tablets are compounded by making a suspension in the mortar and pestle injected through the NG tube. There is sufficient published evidence to suggest that tacrolimus, a lipophilic compound, gets adsorbed onto the NG tubes made up of polyvinyl chloride (PVC). This poses a challenge to the clinician with erratic bioavailability and consequently low tacrolimus concentration. This unintended alteration may lead to graft dysfunction and rejection due to low tacrolimus attained in the systemic circulation.

Further, in clinical situation like acute diarrhoea, there may be rapid absorption of tacrolimus through the denuded intestinal mucosa. This leads to higher than intended tacrolimus peak concentration in the blood and therefore increasing the risk of allograft nephrotoxicity risk and damage to the pancreatic islet cells and development of altered glucose tolerance.

In intravenous (i.v) administration of tacrolimus, a high degree of caution and close monitoring is required (slow infusion with toxicity monitoring) apart from careful dose titration (dose reduction range from ⅓ to ⅕ of oral dose). The intravenous formulation contains castor oil and, therefore, carries a higher risk of anaphylaxis due to excipients. Due to high tacrolimus concentration, bolus infusions may lead to dysrhythmias, QT prolongation, neurotoxicity, and nephrotoxicity. Therefore, the i.v administration of tacrolimus is restricted only to intensive care settings in the USA and some European countries (it is not marketed in India). Even in the USA, about 58% of the centers admitted that they never or rarely prescribe i.v tacrolimus due to fear of toxicity. For patients with diarrhea, i.v tacrolimus is a far-fetched solution because the occurrence of diarrhea is often chronic, and it is not feasible to continue them on injectables, as this drug needs to be administered for throughout the life of the transplanted organ.

Therefore, there is an unmet need in the art to develop a drug delivery system for tacrolimus that is safe, economical and overcomes the challenges of tacrolimus administration to these patients. Moreover, the proposed delivery of tacrolimus is also a viable option for conscious, ambulatory patients as a part of their maintenance immunosuppression because of the rationale mentioned below.

### Objective of the Invention

An objective of the present disclosure aims to deliver tacrolimus through the buccal mucosa route to the systemic circulation (New Route).

Another objective is to provide an efficient delivery with a reduction in administered dose by 30-50% by avoiding the first-pass metabolism of tacrolimus (Reduction in Dose).

Another objective of the present invention is to provide immediate as well as steady concentration of tacrolimus in the systemic circulation via buccomucoadhesive patches, overcoming the concerns of erratic absorption by oral route by modulating the release of the drug (Controlled Release).

Another objective of the present invention is to improve patient compliance by using a specially designed buccal patch applicator which imparts precision to the application process thus avoiding the chances of erroneous application thereby improving patient compliance (Ease of Use).

### SUMMARY OF THE INVENTION

The present disclosure relates to buccal mucoadhesive patch formulation of tacrolimus. The tacrolimus buccal mucoadhesive patch formulation of present invention provides an immediate as well as controlled release of tacrolimus by avoiding the first pass metabolism and attainment of a stable concentration of tacrolimus in the systemic circulation.

The present invention relates to buccal mucoadhesive patch formulation of tacrolimus for buccal administration for systemic use.

In an aspect, the present invention relates to a buccal mucoadhesive patch comprising therapeutically effective amount of tacrolimus loaded on to a solid lipid nanoparticle composition in a carrier polymeric matrix for delivery of the tacrolimus through the trans buccal route to the systemic circulation.

In another aspect of the present invention, the therapeutically effective amount of tacrolimus-loaded solid lipid nanoparticle (SLN) composition comprises,
(a) tacrolimus in an amount ranges from 0.1% to 1% by total weight of the composition,
(b) one or more polymer in an amount ranges from 1% to 5% by total weight of the composition;
(c) one or more pharmaceutically acceptable additives in an amount ranges from 25%-50% by total weight of the composition; and
(d) water in a quantity required.

In another aspect of the present invention, the carrier polymer matrix for delivery of the tacrolimus comprises hydroxypropyl methylcellulose, methyl cellulose and propylene glycol.

In an aspect, the present invention relates to a buccal mucoadhesive patch formulation of tacrolimus comprising of:
(a) therapeutically effective amount of tacrolimus;
(b) polymeric carrier matrix; and
(c) one or more pharmaceutically acceptable additives.

In another aspect of the present invention, the polymeric matrix comprises one or more polymer suitable for preparing an immediate release component and controlled release component of tacrolimus loaded into the patch.

In another aspect of the preparation, the buccal mucoadhesive patch formulation of tacrolimus also comprises a proportion of the total drug in a nanocarrier based formulation of tacrolimus in an optimum ratio.

In another aspect, the present invention provides a method of preparing buccal mucoadhesive patch formulation of tacrolimus comprising of a smaller proportion of immediate release form to provide instant drug concentration in the blood and the larger proportion in form of nanocarrier-based formulation to provide controlled release of the drug into the systemic circulation.

Various objects, features, aspects and advantages of the inventive subject matter will become more apparent from the following detailed description of preferred embodiments.

### BRIEF DESCRIPTION OF THE FIGURES

FIG. 1 shows the graph of the dissolution profile of the buccal patch formulation of the present invention from the beaker dissolution test and USP type-II Paddle Apparatus test.

FIG. 2 shows the in-vitro toxicity of the buccal patch formulation of the present invention against the human keratinocyte cell line and human fibroblast cell line.

FIGS. 3 and 4 show the pharmacokinetics effect of orally administered tacrolimus and buccal patch formulation of the present invention, respectively.

### DETAILED DESCRIPTION OF THE INVENTION

The following is a detailed description of embodiments of the present disclosure. The embodiments are in such detail as to clearly communicate the disclosure. However, the amount of details offered is not intended to limit the anticipated variations of embodiments; on the contrary, the intention is to cover all modifications, equivalents, and alternatives falling within the spirit and scope of the present disclosure as defined by the appended claims.

Unless the context requires otherwise, throughout the specification which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

In some embodiments, the numbers expressing quantities of ingredients, properties such as concentration, process conditions, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

To the extent a term used in a claim is not defined below, it should be given the broadest definition persons in the pertinent art have given that term as reflected in printed publications and issued patents at the time of filing.

As used herein the term “patch” includes films, sheets and wafers, in any shape, including rectangular, square, or other desired shape. The patch described herein may be of any desired thickness and size such that it may be placed into the oral cavity of the user. For example, the patches may have a relatively thin thickness from about 0.1 to about 10 mils, or they may have a thickness varying from 10 to 30 mils. For some patches, the thickness may be even larger, i.e., greater than about 30 mils. Patches may be in a single layer or multi-layered, including laminated patches.

As used herein, a “solid lipid nanoparticle”, or “SLNs”, is a non-vesicular lipid aggregate, having a diameter of less than 1 micrometer (μm) (i.e., less than 1000 nm), that is solid at room temperature. In one embodiment, the SLN of the invention has a diameter of 100-450 nm (Preferably 100-350 nm). As further used herein, a “non-vesicular lipid aggregate” is a lipid structure which does not form a closed internal volume (vesicle); in particular, it is neither a unilamellar nor a multilamellar liposome.

Nano-carrier forms of tacrolimus may be micellar forms or colloidal systems. The present disclosure also relates to formulating microsponges or microspheres loaded with tacrolimus. Amphiphilic micellar carriers are suitable for poorly water-soluble drugs which can be loaded to the hydrophobic core. Microsponges (or microspheres) are rigid, porous and sponge-like round microscopic particles of cross-linked polymer beads each defining a substantially non-collapsible pore network. Microsponges can be loaded with an active ingredient and can provide a controlled time release of the active ingredient to skin or to a mucosal membrane upon application of the formulation.

The present disclosure relates to buccal mucoadhesive patch formulation of tacrolimus for buccal mucosal administration. The tacrolimus buccal mucoadhesive patch formulation of present invention provides an immediate and controlled release of tacrolimus by avoiding the first pass metabolism and providing stable concentration of tacrolimus in the systemic circulation.

In an embodiment, the present invention relates to a buccal mucoadhesive patch therapeutically effective amount of tacrolimus loaded solid lipid nanoparticle composition and a carrier polymeric matrix for delivery of the tacrolimus.

In another embodiment of the present invention, the therapeutically effective amount of tacrolimus loaded solid lipid nanoparticle composition comprises,
(a) tacrolimus in an amount ranges from 0.1% to 1% by total weight of the composition,
(b) one or more polymer in an amount ranges from 1% to 5% by total weight of the composition;
(c) one or more pharmaceutically acceptable additives in an amount ranges from 25%-50% by total weight of the composition; and
(d) water in a quantity required.

In yet another embodiment, the present invention relates to a buccal mucoadhesive patch comprising therapeutically effective amount of tacrolimus loaded solid lipid nanoparticle composition and a carrier polymeric matrix for delivery of the tacrolimus;

wherein:
the therapeutically effective amount of tacrolimus loaded solid lipid nanoparticle composition comprises,
(a) tacrolimus in an amount ranges from 0.1% to 1% by total weight of the composition,
(b) one or more polymer in an amount ranges from 1% to 5% by total weight of the composition;
(c) one or more pharmaceutically acceptable additives in an amount ranges from 25%-50% by total weight of the composition; and
(d) water in a quantity required.

In yet another embodiment, the present invention relates to a buccal mucoadhesive patch therapeutically effective amount of tacrolimus loaded solid lipid nanoparticle composition and a carrier polymeric matrix for delivery of the tacrolimus;

wherein:
the therapeutically effective amount of tacrolimus loaded solid lipid nanoparticle composition comprises,
(a) tacrolimus in an amount ranges from 0.4% to 0.5% by total weight of the composition,
(b) one or more polymer in an amount ranges from 1.5% to 2.5% by total weight of the composition;
(c) one or more pharmaceutically acceptable additives in an amount ranges from 30%-40% by total weight of the composition; and
(d) water in a quantity required.

In yet another embodiment, the present invention relates to a buccal mucoadhesive patch with a therapeutically effective amount of tacrolimus-loaded composition and a carrier polymeric matrix for delivery of the tacrolimus;

wherein:

The therapeutically effective amount of tacrolimus-loaded solid lipid nanoparticle composition comprises,
(a) tacrolimus in an amount of 0.45% by total weight of the composition,
(b) poloxamer in an amount of 1.78% by total weight of the composition;
(c) tween-80 in an amount of 16.8% by total weight of the composition; and
(d) water to make total weight of the composition to 100%.

In yet another embodiment, the present invention relates to a buccal mucoadhesive patch therapeutically effective amount of tacrolimus loaded solid lipid nanoparticle composition and a carrier polymeric matrix for delivery of the tacrolimus comprises hydroxypropyl methylcellulose (HPMC E5), methyl cellulose and propylene glycol;

wherein:
the therapeutically effective amount of tacrolimus loaded solid lipid nanoparticle composition comprises,
(a) tacrolimus in an amount of 0.45% by total weight of the composition,
(b) poloxamer in an amount of 1.78% by total weight of the composition;
(c) tween-80 in an amount of 16.8% by total weight of the composition; and
(d) water to make total weight of the composition to 100%.

In yet another embodiment, the present invention relates to a buccal mucoadhesive patch therapeutically effective amount of tacrolimus loaded solid lipid nanoparticle composition and a carrier polymeric matrix for delivery of the tacrolimus comprises hydroxypropyl methylcellulose (HPMC E5) in an amount ranges from 8%-10% by total weight of the carrier polymer matrix, methyl cellulose in an amount ranges from 1%-2% by total weight of the polymer matrix and propylene glycol in an amount ranges from 4%-5% by total weight of the polymer matrix;

wherein:
the therapeutically effective amount of tacrolimus loaded solid lipid nanoparticle composition comprises,
(a) tacrolimus in an amount of 0.45% by total weight of the composition,
(b) poloxamer in an amount of 1.78% by total weight of the composition;
(c) tween-80 in an amount of 16.8% by total weight of the composition; and
(d) water to make total weight of the composition to 100%.

In an embodiment, the present invention relates to a buccal mucoadhesive patch formulation of tacrolimus comprising of:
(a) therapeutically effective amount of tacrolimus;
(b) polymeric carrier matrix; and
(c) one or more pharmaceutically acceptable additives.

In an embodiment of the present invention, the tacrolimus is present in an amount ranges from 0.5 mg to 3.0 mg.

In another embodiment of the present invention, the carrier polymer matrix may be of any desired polymeric carrier matrix having oral solubility. The buccal mucoadhesive patches are preferably moderate-dissolving in the oral cavity and particularly suitable for delivery of actives, both fast and controlled release compositions.

In another embodiment of the present invention, the polymer included in the patches may be water-soluble polymer, water-swellable polymer, or a combination of one or more either water-soluble, or water-swellable polymers.

In various embodiments, the polymer may include polyethylene oxide, polyvinylpyrrolidone, cellulose, a cellulose derivative, polyvinyl alcohol, a polysaccharide, and combinations thereof. Specific examples of useful water-soluble polymers include, but are not limited to, polyethylene oxide, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl copolymers, starch, gelatin, and combinations thereof. The water-swellable polymer includes the polyacrylamide, sodium carboxymethyl cellulose (NaCMC) or hydroxypropyl methyl cellulose (HPMC) or combination thereof.

In another embodiment of the present invention, the polymer in the solid lipid nanoparticle composition is poloxamer, and the pharmaceutically acceptable additive is glyceryl dibehenate (Compritol-888 ATO) or polysorbate (tween-80).

In another embodiment of the present invention, the pharmaceutically acceptable additives include but not limited to a buffering agent, an artificial sweetener, colouring agent, flavouring agent, penetration enhancer, plasticizers, tonicity agents and preservatives.

In another embodiment of the present invention, the buffering agent can be selected from the group consisting of an amino acid, an alkali metal salt of an amino acid, aluminum hydroxide, aluminum hydroxide/magnesium carbonate/calcium carbonate co-precipitate, aluminum magnesium hydroxide, aluminum hydroxide/magnesium hydroxide co-precipitate, aluminum hydroxide/sodium bicarbonate coprecipitate, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, trometamol, and mixtures thereof.

In another embodiment of the present invention, the artificial sweetener is selected from the group consisting of aspartame, acesulfame K, saccharin, sucralose, altitame, cyclamic acid and its salts, glycerrhizinate, dihydrochalcones, thaumatin, monellin, or any other non-cariogenic, sugar-free sweetener may be used, alone or in combination.

In another embodiment of the present invention, the coloruing agent is selected from the group consisting of di-iron trioxide, yellow ferric oxide, famille rose, caramel, beta carotene, phosphorus sour riboflavin sodium, and aluminum lake.

In another embodiment of the present invention, the flavouring agent is selected from group consisting of mint, peppermint, strawberry and Tuttifrutti.

In another embodiment of the present invention, the penetration enhancer is selected from the group consisting of phospholipids, sucrose esters of fatty acids, polyoxyl stearate, polyoxyethylene hydrogenated castor oil, polyoxyethylene polyoxypropylene glycol, sorbitan sesquioleate, sorbitan trioleate, sorbitan monostearate, sorbitan monopalmitate, sorbitan monolaurate, polysorbate, glyceryl monostearate, sodium lauryl sulfate, sodium dodecyl sulfate, lauromacrogol Arlasolve, Poloxamers, Labrafil, Labrasol, Tween 80 and the like.

In another embodiment of the present invention, the plasticizers is selected from the group consisting of citrate esters (e.g., triethylcitrate, triacetin), low molecular weight polyalkylene oxides (e.g., polyethylene glycols, polypropylene glycols, polyethylene/propylene glycols), glycerol, pentaerythritol, glycerol monoacetate, diacetate or triacetate, propylene glycol, and sodium diethyl sulfosuccinate.

In another embodiment of the present invention, the tonicity agent is selected from the group consisting of glycerin, lactose, mannitol, dextrose, sodium chloride, sodium sulfate, sorbitol, saline-sodium citrate (SSC), and the like.

In another embodiment of the present invention, the preservative can be selected from the group consisting of benzyl alcohol, methyl paraben, propyl paraben, thiomerosol, phenylmercury salts (phenylmercuric acetate, phenylmercuric borate, phenylmercuric nitrate), chlorobutanol, metacresol, and the like.

In one or more embodiments the patch of the present invention comprises tacrolimus loaded into the nanocarriers.

In another embodiment of the present invention, the DSC and FTIR studies confirmed the incorporation of drugs into the lipidic matrix as no separate peak was observed.

In another embodiment of the present invention, the particle size of tacrolimus in solid lipid nanoparticles is in a range of 100 nm to 350 nm.

In another embodiment of the present invention, the polydispersity index (PDI) of tacrolimus loaded solid lipid nanoparticles is in a range of 0.1 to 0.3.

In another embodiment of the present invention, the Zeta potential of tacrolimus loaded solid lipid nanoparticles is +30 mV to −30 mV.

In another embodiment of the present invention, the tacrolimus loaded SLN composition has following properties.

| Parameter | Optimized values | |
|---|---|---|
| Concentration of drug | 0.8% | w/w |
| Average particle size | 250 ± 25 | nm |
| PDI | <0.300 | |
| Zeta potential | +30 to −30 | mV |

| | |
|---|---|
| Total Drug Content | 91-109% |
| Entrapment Efficacy | 84-86.9% |
| Free drug | 10-18% |

In another embodiment, the buccal mucoadhesive patch formulation of the present invention overcomes the poor aqueous solubility of tacrolimus providing higher drug loading and allow efficient mucosal crossing, overcome the likely dose dumping that may occur if 100% of the drug in the buccal mucoadhesive patch is immediate release (IR) form.

In another embodiment of the present invention, the nanocarrier form of tacrolimus provides a controlled release for obtaining a steady concentration of tacrolimus in systemic circulation.

In another embodiment, the buccal mucoadhesive patch formulation of the present invention provides controlled and efficient release of tacrolimus over the dosing interval of 12 hours without producing high Cₘₐₓ, which produces unpleasant symptoms like dizziness and tremor as complained by many of our patients during the first hour of oral intake of the capsules in immediate release form.

In another embodiment, the buccal mucoadhesive patch formulation of the present invention provides therapeutically effective concentration for a duration of 12 hours via delivering the drug in a phased manner, i.e., IR component and the CR component.

In another embodiment of the present invention, the time taken for the system to deliver the drug systemically will be less than 30 minutes thereby allowing ease of administration for a comatose or intubated patient as well as improve the compliance of an ambulatory patient.

In an embodiment of the present invention, the buccal mucoadhesive patch has a small size that is between about 0.5-1 inch by about 0.5-1 inch. Most preferably, the patch size is about 0.75 inches×0.5 inches. The patch should have good adhesion when placed in the buccal cavity of the user. Further, the patch should disperse and dissolve at a rate, that is, between about 1 minute to about 30 minutes, and most desirably between about 10 minutes and about 20 minutes.

In another embodiment of the present invention, the buccal mucoadhesive patch formulation of tacrolimus is completely dissolvable in nature in the buccal cavity and there is no need to peel off.

In another embodiment of the present invention, the buccal patch formulation of tacrolimus (BAPTac) has a mean AUC₀₋₂₄ of 512.0225 ng/ml*hour.

In another embodiment of the present invention, the buccal patch tacrolimus (BAPTac) of the present invention shows significantly higher exposure of the drug over a 24-hour period than the oral route, providing the proof of concept. The tacrolimus buccal patch of the present invention showed doubled AUC₀₋₂₄ when compared to oral tacrolimus suggesting that the BAPTac may reduce the dose by 40-50% and provide sustained drug release over the whole dosing interval.

In another embodiment of the present invention, the buccal patch tacrolimus (BAPTac) of the present invention shows the controlled release of the tacrolimus for a period of 12 hours or more.

In another embodiment, the buccal patch tacrolimus (BAPTac) of the present invention provides the sustained release of the tacrolimus for a period of 12-hour or more.

In yet another embodiment, the buccal patch tacrolimus (BAPTac) of the present invention can be administered twice a day (b.d or b.i.d).

In yet another embodiment, the buccal patch tacrolimus (BAPTac) of the present invention can be administered twice a day (b.d or b.i.d) or once a day (o.d).

In another embodiment, the present invention provides a method of preparation of buccal mucoadhesive patch comprising the steps of:
(a) preparing tacrolimus loaded solid lipid nanoparticle composition comprising therapeutically effective amount of tacrolimus;
(b) adding concentrated tacrolimus loaded solid lipid nanoparticle to a polymeric matrix;
(c) adding triacetin drop wise to the polymer matrix containing the concentrated SLN of tacrolimus to increase homogeneity; for obtaining a rheologically acceptable formulation;
(d) casting the buccal mucoadhesive patch by pouring the mixture obtained from step (c) on to the release liner (Table Top Film Forming Machine, VJTDP-Lab) to obtain buccal mucoadhesive patch.

In another embodiment of the present invention, the patch of the present invention can be produced by a combination of at least one polymer and a solvent, optionally including other fillers known in the art. Examples of solvent include but not limited to water, a polar organic solvent such as ethanol, isopropanol, acetone, or any combination thereof.

While the foregoing description discloses various embodiments of the disclosure, other and further embodiments of the disclosure may be devised without departing from the basic scope of the disclosure. The invention is not limited to the described embodiments, versions or examples, which are included to enable a person having ordinary skill in the art to make and use the invention when combined with information and knowledge available to the person having ordinary skill in the art.

### EXAMPLES

The present disclosure is further explained in the form of following examples. However, it is to be understood that the foregoing examples are merely illustrative and are not to be taken as limitations upon the scope of the invention. Various changes and modifications to the disclosed embodiments will be apparent to those skilled in the art.

### Example-1

| S. No | Ingredients | Amount | |
|---|---|---|---|
| 1 | Tacrolimus | 0.5 to 3.0 | mg |
| 2 | HPMC E5 (12.5%) | 5 | g |
| 3 | PVP K −30 (4%) | 1.6 | g |
| 3 | Glycerol | 2 | ml |
| 5 | Water | 40 | ml |

Glycerol (2 ml) was added to water (37 ml). The whole volume was divided into three parts. One part of the solution was warmed up and then HPMC E5 was added to it gradually on the magnetic stirrer and this continued for about 30 minutes. The solution was cooled to room temperature and PVPK-30 was added after adding the rest of the ⅔ of the solvents. The mixture was stirred over the magnetic stirrer for another 30 minutes at ambient temperature.

Tacrolimus stock solution was prepared in polypropylene carbonate with dichloromethane. Then 1 ml of the drug stock solution was added to the polymer mixture and sonicated for 30 minutes. Thereafter, it was retained overnight at 4° C. in the refrigerator. The film was casted on the table top film forming machine on the base film. After the films were half dry, the backing film was attached.

Examples 2-4: The following examples are prepared by following the above experimental procedures with non-critical variations.

| Ingredients | Ex - 2 | | Ex - 3 | | Ex - 4 | |
|---|---|---|---|---|---|---|
| Tacrolimus | 0.5 to 3.0 | mg | 0.5 to 3.0 | mg | 0.5 to 3.0 | mg |
| HPMC E5 (12.5%) | 7.5 | g | 1.8 | g | 6 | g |
| Kollicoat IR (5%) | 3 | g | — | | — | |
| PEO 0.5% | 300 | mg | — | | — | |
| PVA | — | | 6 | g | — | |
| PVP K −30 (4%) | — | | 3 | g | — | |
| Methyl cellulose | — | | — | | 2.4 | g |
| Propylene glycol | — | | — | | 4 | ml |
| Glycerol | 7 | ml | 7 | ml | — | |
| Water | 60 | ml | 60 | ml | 60 | ml |

### Example 5

| | | Weight | |
|---|---|---|---|
| S. No | Ingredients | (g) | w/w % |
| 1 | Tacrolimus API | 0.15 | 0.45 |
| 2 | Tween-80 (Polysorbate) | 9 | 26.8 |
| 3 | Poloxamer-188 | 0.6 | 1.8 |
| 4 | Compritol-888 ATO (Glyceryl Dibehenate) | 2.18 | 6.5 |
| 5 | Water | Qs | q.s |

### Method of Preparing Tacrolimus Loaded SLN

Step 1: Tacrolimus was mixed with tween 80 and afterwards poloxamer 188 and water is added, mixed and heated to 70° C. to obtain component A.
Step 2: Compritol 888 ATO is kept in another beaker and is heated to 70° C. to obtain component B.
Step 3: Added component B to component A at 70° C. under continuous stirring on magnetic stirrer for 10 minutes to obtain hot micro-emulsion.
Step 4: Dispersed the hot micro-emulsion into chilled water (0-4° C.) with vigorous stirring at 13000 rpm using IKA high pressure homogenizer for 45 minutes of 15 min cycles each to obtain tacrolimus loaded solid lipid nanoparticles.

### Procedure for Loading Tacrolimus Solid Lipid Nanoparticles to the Buccal Mucoadhesive Patch

| S. No | Ingredients | Weight (g) |
|---|---|---|
| 1 | Tacrolimus loaded SLN (example 5) | 10 ml |
| 2 | Polymer Matrix | |
| | HPMC E5 | 10 |
| | Methyl cellulose | 2 |
| | Propylene glycol | 5.2 |
| | Water | q.s |

Step 1: Weighed the SLN formulation (10 ml) in order to load into the buccal mucoadhesive patch.
Step 2: Added the concentrated tacrolimus loaded solid lipid nanoparticle to a polymeric matrix.
Step 3: Added triacetin drop wise to the polymer matrix containing the concentrated SLN of tacrolimus to increase homogeneity for obtaining a rheologically acceptable formulation.
Step 4: Casted buccal mucoadhesive patch by pouring the mixture obtained from step (c) on to the release liner (Table Top Film Forming Machine, VJTDP-Lab).

### Characterization of the SLN Loaded Buccal Patch

### In-Vitro Dissolution Data

### Beaker Dissolution Test

Doses tested: 1 mg BAPTac patch
Dissolution fluid: Phosphate Buffered Saline with 30% Ethanol
Volume: 50 ml
   The results are tabulated below.

| Time | | | | | |
|---|---|---|---|---|---|
| point | | | | Average Conc. | % Drug release |
| (min) | Set A | Set B | Set C | (ug) Mean ± SD | Mean ± SD |
| 5 | 122.84 | 111.67 | 209.36 | 148 ± 53.47 | 14.79 ± 5.34 |
| 10 | 358.97 | 390.88 | 475.33 | 408.4 ± 60.12 | 40.84 ± 6.01 |
| 15 | 701.82 | 638.02 | 728.03 | 689.3 ± 46.29 | 68.92 ± 4.63 |
| 30 | 857.35 | 779.41 | 868.25 | 835 ± 48.45 | 83.50 ± 0.27 |
| 45 | 942.84 | 1020.67 | 949.88 | 971.1 ± 43.64 | 97.11 ± 1.52 |

### (b) Dissolution Test in USP Type-II Paddle Apparatus

Doses tested: 3 mg BAPTac patch
Dissolution fluid: Phosphate Buffered Saline with 30% Ethanol
Volume: 500 ml

| Time | Set A | Set B | Set C | Average (% |
|---|---|---|---|---|
| point | (% Drug | (% Drug | (% Drug | Drug release) |
| (min) | release) | release) | release) | Mean ± SD |
| 5 | 18.07 | 14.84 | 18.06 | 16.99 ± 1.86 |
| 10 | 21.54 | 28.53 | 32.54 | 27.54 ± 5.57 |
| 15 | 51.04 | 44.03 | 46.04 | 47.03 ± 3.59 |
| 30 | 91.18 | 91.68 | 78.90 | 87.26 ± 7.24 |
| 45 | 97.78 | 98.37 | 96.69 | 97.61 ± 0.85 |
| 60 | 100.00 | 100.00 | 100.00 | 100.00 |

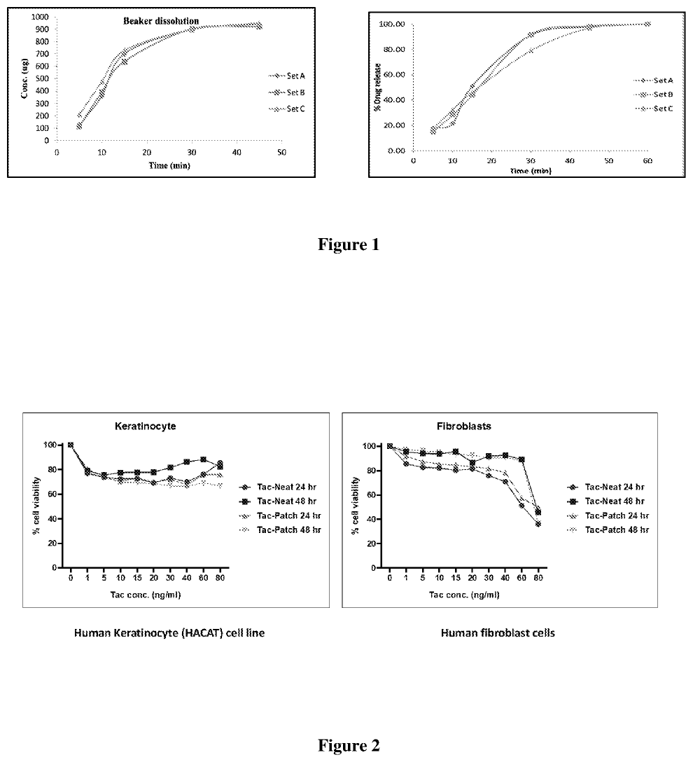

FIG. 1 shows the graph of the dissolution profile from beaker dissolution test and USP type-II Paddle Apparatus test. The dissolution data clearly indicates that the amount of drug released within 60 minutes was in confirmation with an amount loaded in lipidic nanoparticles.

### Stability Testing

The stability studies were conducted under room temperature (25-35° C., RH 65±10%) and accelerated conditions (40±2.0° C., RH 75±5%) The results are provided in the table below.

| | Time | Average | Total drug | Entrapment |
|---|---|---|---|---|
| | points | particle | content | efficiency |
| | (months) | size (nm) | (%) | (%) |
| At room temp | 0 | 249.86 | 98.27 ± 1.16 | 86.91 ± 2.16 |
| (25-35° C., RH | 1 | 271.27 | 99.98 ± 1.73 | 84.4 ± 2.81 |
| 65 ± 10%) | 2 | 242.57 | 97.34 ± 1.28 | 81.97 ± 1.53 |
| | 3 | 251.32 | 96.78 ± 1.82 | 80.19 ± 2.62 |
| Accelerated | 0 | 249.86 | 98.27 ± 1.16 | 86.91 ± 2.16 |
| stability | 1 | 248.12 | 96.08 ± 2.56 | 84.23 ± 1.78 |
| 40 ± 2.0° C. | 2 | 245.71 | 95.32 ± 2.55 | 84.19 ± 3.18 |
| And RH 75 ± 5% | 3 | 245.93 | 92.87 ± 1.12 | 81.71 ± 2.86 |
| In the stability | | | | |
| Chamber | | | | |

The stability studies demonstrated that the buccal patch formulation of tacrolimus is stable up to three months.

### In-Vitro Toxicity

MTT (3-[4,5-dimethylthiazol-2-yl]-2,5 diphenyl tetrazolium bromide) assay was performed per current best practices and procedures.
Cells: Cell lines of human origin (1) Fibroblasts (2) Keratinocytes
Treatment: Group 1-Neat (unchanged) tacrolimus Group 2-Tacrolimus SLNs in 1% DMSO
Concentrations: 0, 1, 5, 10, 15, 20, 30, 40, 50, 60 and 80 ng/ml
Duration of treatment: 24 hours, 48 hours

FIG. 2 demonstrates the toxicity effect on human keratinocyte cell line and human fibroblasts cell line. The buccal patches of the present invention are found to be safe in in-vitro settings.

### In-Vivo Pharmacokinetic Evaluation

The study was performed in New Zealand White rabbits as per the CPCSEA guidance. Overnight fasted animals were anaesthetised with Ketamine (30-40 mg/kg) and Xylazine (10 mg/kg) and 1 mg BAPTac patch was administered on the buccal surface of the oral cavity. The control group was administered 1 mg tacrolimus in PBS and 30% Ethanol. 0.5-0.75 ml of blood was taken from each rabbit via the marginal ear vein using 23 gauge needles through a butterfly cannula. The blood samples were collected before initiation of the treatment and after the administration of oral Tacrolimus or Tacrolimus-SLN-loaded buccal patch at time intervals of 15 min, 30 min, 1 h, 1.5 h, 2 h, 4 h, 6 h, 8 h, 12 h and 24 hours for the single dose PK study. Blood samples were collected in EDTA tubes, aliquoted and stored in amber-coloured tubes at −80° C. until quantification by LC-MS/MS. The results are tabulated below.

| | | Oral Tacrolimus | | BAPTac | |
|---|---|---|---|---|---|
| Sample | | Dosing Group | | Dosing Group | |
| collection | | Average Tac | | Average Tac | |
| time in (mins) | | conc. (ng/μl) | SD | conc. (ng/μl) | SD |
| 0 | | 0 | 0 | 0 | 0 |
| 0.25 | (15 mins) | 3.6 | 0.4 | 17.7 | 3.5 |
| 0.5 | (30 mins) | 12.3 | 2.8 | 45.3 | 10.9 |
| 1.0 | (60 mins) | 17.2 | 4.6 | 29.4 | 5.6 |
| 1.5 | (90 mins) | 46.9 | 8.9 | 29.4 | 6.2 |
| 2.0 | (120 mins) | 31.3 | 5.9 | 29.6 | 6.5 |
| 4.0 | (240 mins) | 14.5 | 4.1 | 28.4 | 5.1 |
| 6.0 | (480 mins) | 12.3 | 1.5 | 33 | 9.2 |
| 8.0 | (640 mins) | 11.8 | 2.1 | 23.4 | 6.5 |
| 12.0 | (720 mins) | 7.9 | 1.81 | 19.2 | 5.2 |
| 24.0 | (1440 mins) | 2.4 | 0.48 | 9.8 | 1.9 |

| | | | | |
|---|---|---|---|---|
| Mean AUC₀₋₂₄ | 250.38 ± 78.23 | ng/ml · hr | 512.02 ± 105.45 | ng/ml · hr |
| Elimination t_{1/2} | 2.73 ± 2.9 | hr | 45.96 ± 10.38 | hr |

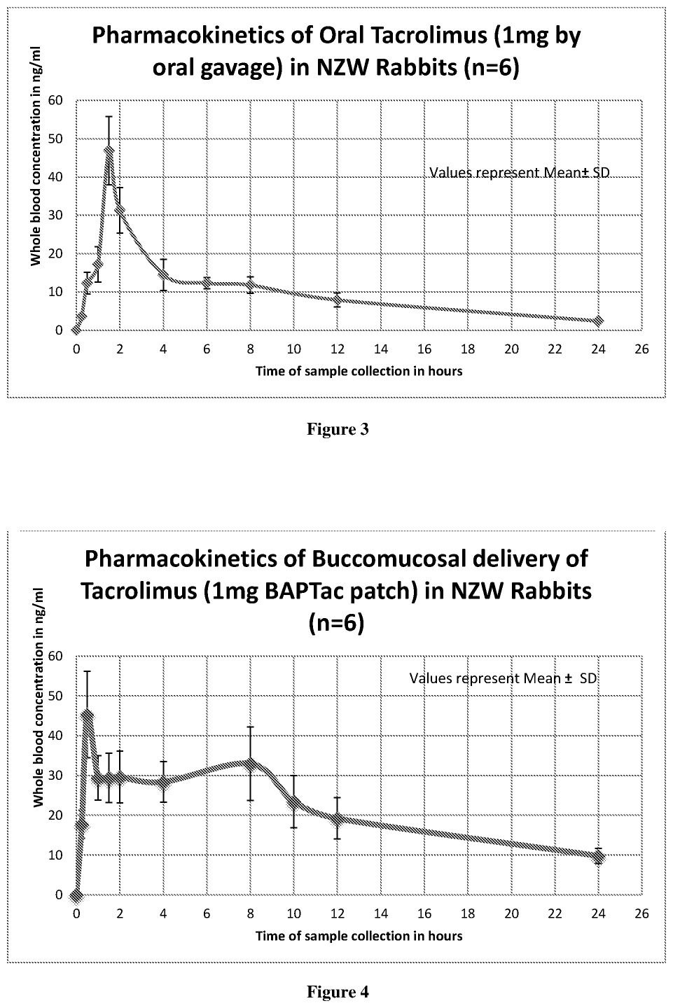

FIGS. 3 and 4 depict the PK data of oral tacrolimus and buccal mucoadhesive patch tacrolimus, respectively. It was found that the exposure of the drug over 24-hour period with buccal patch formulation of tacrolimus (BAPTac) is significantly higher than the oral route, providing the proof of concept. The nearly doubled AUC₀₋₂₄ with BAPTac compared to oral tacrolimus suggests that the BAPTac may reduce the dose by 40-50% and provide sustained drug release over the whole dosing interval.

A skilled artisan will appreciate that the quantity and type of each ingredient can be used in different combinations or singly. All such variations and combinations would be falling within the scope of present disclosure.

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### Advantages of the Present Invention

The present invention provides a tacrolimus buccal mucoadhesive patch having significantly higher exposure of the drug over 24-hour period than the oral route.

The present invention provides a tacrolimus buccal mucoadhesive patch having doubled AUC₀₋₂₄ compared to oral tacrolimus suggesting that the dose may be reduced by 40-50%.

The present invention provides a tacrolimus buccal mucoadhesive patch having sustained drug release over the whole dosing interval.

### REFERENCES

1. Pennington C A, Park J M. Sublingual tacrolimus as an alternative to oral administration for solid organ transplant recipients. Am J Health Syst Pharm. 2015 Feb. 15; 72 (4): 277-84. doi: 10.2146/ajhp140322.
2. Hochleitner B-W, Bosmiiller C, Nehoda H, Friihwirt M, Simma B, Ellemunter H, Steurer W, Hochleitner E-O, Konigsrainer A, Margreiter R. Increased tacrolimus levels during diarrhea. Transpl Int (2001) 14:230-233.
3. Doligalski C T, Liu E C, Sammons C M, Silverman A, Logan A T. Sublingual administration of tacrolimus: current trends and available evidence. Pharmacotherapy: The Journal of Human Pharmacology and Drug Therapy. 2014 November; 34 (11): 1209-19.
4. Romero I, Jimémez C, Gil F et al. Sublingual administration of tacrolimus in a renal transplant patient. J Clin Pharm Ther. 2008; 33:87-9.
5. Tsapepas D, Saal S, Benkert S et al. Sublingual tacrolimus: a pharmacokinetic evaluation pilot study. Pharmacotherapy. 2013; 33:31-7.
6. Harris M T, Collins B H, Vikraman D, et al. Sublingual tacrolimus after high risk kidney transplant. Presented in abstract format at the American Transplant Congress, 2010.
7. Solari S, Cancino A, Wolff R, Norero B, Vargas J I, Barrera F, Guerra J F, Martinez J, Jarufe N, Soza A, Arrese M, Benite C. Sublingual tacrolimus administration provides similar drug exposure to per-oral route employing lower doses in liver transplantation: a pilot study. Aliment Pharmacol Ther 2017; 45:1225-1231.
8. Federico S, Rosa C, Sabbatini M, Nappi R, Russo L, Apicella L, Balletta M M, Santangelo M, Mosca T, Tarantino G, Capone D. Sublingual administration improves systemic exposure of tacrolimus in kidney transplant recipients: comparison with oral administration. Eur J Clin Invest 2016; DOI: 10.1111/eci.12644.
9. Srinivas N R. Letter: Sublingual dosing of tacrolimus in transplant patients-interesting concept to overcome first pass effects. Aliment Pharmacol Ther. 2017; 46:79-8.
10. Oleary J G. Editorial: Tacrolimus-How low can we go. Aliment Pharmacol Ther 2017; 45:66-67.
11. Bhalodia R (2010) Bucoadhesive drug delivery system: A review. Int J of Pharma and Bio. Sci 1:1-32.
12. Parthasarathy G, Bhaskar K, Jayaveera K N, Prasanth V (2011) Buccal Mucosa: a Gifted Choice for Systemic Drug Delivery. Int J of Drug Deli 3:586-596.

I claim:

1. A buccal mucoadhesive patch comprising of therapeutically effective amount of tacrolimus-loaded solid lipid nanoparticle composition and a carrier polymer matrix for delivery of the tacrolimus.

2. The buccal mucoadhesive patch as claimed in claim 1, wherein the therapeutically effective amount of tacrolimus loaded solid lipid nanoparticle composition comprises,
(a) tacrolimus in an amount ranges from 0.1% to 1% by total weight of the composition,
(b) one or more polymer in an amount ranges from 1% to 5% by total weight of the composition,
(c) one or more pharmaceutically acceptable additives in an amount ranges from 25%-50% by total weight of the composition, and
(d) water in a quantity required.

3. The buccal mucoadhesive patch as claimed in claim 1, wherein the tacrolimus is present in a dose range of 0.5 mg to 3 mg.

4. The buccal mucoadhesive patch as claimed in claim 1, wherein the carrier polymer matrix comprises one or more polymer suitable for preparing an immediate release component and controlled release component of tacrolimus loaded into the patch.

5. The buccal mucoadhesive patch as claimed in claim 2, wherein the one or more polymer is selected from the group consisting of water-soluble polymer, water-swellable polymer, or a combination of one or more either water-soluble, or water-swellable polymers.

6. The buccal mucoadhesive patch as claimed in claim 1, wherein the carrier polymer matrix for delivery of the tacrolimus comprises hydroxypropyl methylcellulose, methyl cellulose and propylene glycol.

7. The buccal mucoadhesive patch as claimed in claim 2, wherein the one or more pharmaceutically acceptable additive is selected from the group consisting of a buffering agent, an artificial sweetener, colouring agent, flavouring agent, penetration enhancer, plasticizers, tonicity agents and preservatives.

8. The buccal mucoadhesive patch composition as claimed in claim 1, wherein particle size of the tacrolimus in solid lipid nanoparticle is in a range of 100 nm to 350 nm.

9. The buccal mucoadhesive patch as claimed in claim 2, wherein the polymer is poloxamer and the one or more pharmaceutically acceptable additive is polysorbate and glyceryl dibehenate.

10. A method of preparation of buccal mucoadhesive patch as claimed in claim 1 comprising the steps of:
(a) preparing tacrolimus loaded solid lipid nanoparticle composition comprising therapeutically effective amount of tacrolimus as claimed in claim 2;
(b) adding concentrated tacrolimus loaded solid lipid nanoparticle to a polymeric matrix;
(c) adding triacetin drop wise to the polymer matrix containing the concentrated SLN of tacrolimus to increase homogeneity for obtaining a rheologically acceptable formulation; and
(d) casting buccal mucoadhesive patch by pouring the mixture obtained from step (c) on to the release liner (Table Top Film Forming Machine, VJTDP-Lab).
